(19) United States
(12) Patent Application Publication
(10) Pub. No.: US 2010/0132133 A1
(43) Pub. Date: Jun. 3, 2010

(54) KERATIN REACTING POLYMERIC STRUCTURES, THEIR SYNTHESIS AND USE

(76) Inventors: Matteo Zanotti Russo, Genova (IT); Nicola Barbini, Milano (IT)

Correspondence Address: Lonnie R Drayer, 6504 Cathedral Oaks Drive, Plant City, FL, 33565, US

(21) Appl. No.: 12/599,293

(22) PCT Filed: May 12, 2008
(86) PCT No.: PCT/IB2008/001177

§ 371 Date: Dec. 10, 2009

(30) Foreign Application Priority Data
May 10, 2007 (EP) 07425276.8

(51) Int. Cl. A61K 8/81; C08F 228/02; C08F 226/02; C08F 218/00; A61Q 5/10

(52) U.S. Cl. 8/428; 526/287; 526/307.6; 526/307.7

(57) ABSTRACT

A (co)polymer structure comprising at least one portion of an acrylic, silicone, polyglycolic, polysaccharide or natural polymeric chain, comprising a plurality of functionalized units of general formula (I): wherein [C-A] is a portion of a polymeric or copolymeric chain selected from an acrylic silicone, polyglycolic, polysaccharide polymeric structure or from a natural polymer, and X is a functional group selected from one or more solfonic, carbossilic, amino acidic or epoxide groups to react at room temperature and in reaction conditions suitable for use on living beings and to form ionic or covalent bonds with keratin in absence of preemptive modifications of the polymer chain itself.

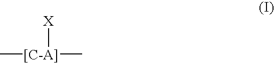

### FIELD OF THE INVENTION

The present invention concerns polymeric structures reacting with keratin, their synthesis, application and use.

The present invention concerns more specifically new (co)polymer structures which present functional groups that make it possible, under safe conditions for the use on living beings and in particular for the use on human beings, to form chemical, ionic or covalent bonds with human keratin, and in particular with the keratin forming hair, eyelashes and eyebrows, skin, nails, lips, oral mucosa and tissues of external genitals organs; the invention also concerns the synthesis of such (co)polymer structures and their use in the cosmetic field.

### BACKGROUND OF THE INVENTION

In recent years many researches connected to products for nails, hair and skin have identified among their primary aims the necessity to implement interactions of materials, substrates, active principles or dyes with keratin matrices which, with some differences, relate body external surfaces and hair. Many cosmetic functions are in fact connected both with change of surface aspects and with the necessity of carrying or fixing onto the keratin surface certain functional structures with both mechanical and chemical function. Among the functions object of such development, the following can be cited:
filmogenic power of hair conditioners
fixatives for hair styling
protective filmogens for hair, skin, nails
water- and abrasion-resistance of suntan products
fixative power of dyes and pigments on nails (enamels), hair (dyes), skin (make-up).

Most of the products intended for nails have the purpose of coloring or applying supporting structures to strengthen the nails; the products intended for skin and hair have instead different functions and objectives. In the trichological field, the use of filmogens has been developed with the express purpose of increasing the durability of the structures applied to the keratin surface as much as possible, as well as in the case of skin products, water-resistant suntan products or ‘barrier effect’ creams have been developed.

WO2007/072521, which was filed before but published after priority date of present application, in the name of the applicants, describes and claims polymers which have functional groups selected from —SH and leaving groups which can be substituted by NH₂ groups present on keratin. The use of —SH groups has the disadvantage to require a preventive change of keratin structure by breaking the disulfide bonds, the use of leaving groups for nucleophilic substitution requires instead reaction conditions which are generally too drastic.

FR2760359 discloses a method for treatment of hair fibers comprising the steps of reducing disulfide bonds of hair keratin to create —SH reactive sites and for the subsequent fixing, with covalent bonds onto said reactive sites, of at least an active compound, such as dyes, reinforcing agents, from external factors protective agents, etc.

US2005/0268405 relates to a compound to dye keratinous fibers, which contains pigments and two kind of polymers (PA+PB), each of them having at least a functional group (A or B) as —COOH and epoxide groups, the two groups being able to form covalent bonds. After application on hair the two polymers react and reaction between the two polymers (PA and PB) produces a insoluble reticulated polymer, which collects on keratinous fibers. U.S. Pat. No. 5,935,560 concerns a compound comprising a silicone polymer functionalized with —SH groups to bind itself with hair fibers by means of covalent bonds. Thiol groups (0, 1 mole %-3 mole %) give a reactive group to bind in covalent way the functionalized silicon with thiol group to the hair. The compound can also contain amine groups (—NH2) which provide further sites to bind silicone to hair and give conditioning features to hair.

EP1192932 describes the development of hair fixative gel for hairstyling with a greater fixative-durability and a greater resistance to humidity. Such performance is achieved because of the filmogenic characteristics of the polymer and because of the interaction between the structure of the polymer and the keratin surface. The filmogenic power increases the contact surface between polymer and keratin, and increases the effectiveness of the Van der Waals bonds and dipole-dipole interactions or hydrogen bonds which ensure the adhesion of the polymer.

US2004146471 concerns a composition having high fixative power and high resistance by way of the use of three filmogenic polymers.

The need to fix and functionalize polymeric substrates is described in EP1525877, which claims the ability to fixing an hair style and to maintain it for a long time thanks to the use of polymers whose structure changes according to temperature.

US2003135004 concerns a composition of polymers which thanks to their particular structure ensure adhesion and durability by their interaction with keratin; their use is described for fixing dyes on face skin (for use in makeup products), on nails, hair, eyelashes or eyebrows.

The use of silicon polymers containing —SH groups to react temporarily with the structure of hair has been known for many years: examples of such polymers are mentioned in GB-A-1199776, EP-A-0295780, EP-A-0437099 and U.S. Pat. No. 5,935,560.

Among the functional principles that are generally attempted to be attached to keratin substrates are pigments and generally coloring substances. The coloration of cosmetic products as well as the ability of cosmetic products to color skin, nails and hair are regulated in Europe, USA and in most countries, therefore the permitted coloring agents are described in explicit lists, as described in Attachments to the Italian L.713/86 and to the European Directive 76/768/EEC. Because it was not easy to intervene for modifying pigment agents, research has therefore been directed into substrates, mostly filmogens, that increase pigment agent anchorage and abrasion-resistance, as described in EP0960617.

EP1321126 describes and claims the use of two complementary polymers that react to form covalent bonds between themselves only when they are distributed on keratin, thus constituting a film that is extremely resistant to rinsing and abrasion. The anchorage to keratin in this case is due to the formation of efficient in situ macromolecular structures that allow to increase interactions with keratin and consequently no-covalent adhesion.

In WO2004098488 water-soluble dithiols comprising a C₃-C₁₀ chain between the two thiol groups are employed as substitution reducing agent of thioglycolic acid and its derivatives, with the principal advantage of achieving a “permanent” effect (breaking the disulfide bonds present in keratin and re-establishing them in the “perm”) under favorable pH conditions and with not very odorous reagents.

The problem with the above described formulations and the systems is that the structures produced have a limited lifetime, and in some cases they do not withstand washing. There is therefore the need to provide formulations for use with hair that can be able to have a longer useful life than is now possible.

### SUMMARY OF THE INVENTION

A purpose of the present invention is to resolve the problem discussed above and to produce formulations for the treatment of hair in particular, and of hair, nails and skin in general, able to produce a lasting effect.

Such purpose is achieved by the present invention that relates to a (co)polymer for cosmetic use, characterized according to claim 1.

More specifically, an object of the present invention is a class of (co)polymers applicable in the cosmetic sector and in particular in the formulation of products for the care and treatment of hair, eyelashes, eyebrows, body-hair, nails mucosae and skin, which are characterized by the presence of reactive functional groups that are able to produce chemical bonds (ionic or covalent) with the keratin under mild conditions (compatible with the necessary safety requirements for their use on humans) and in the presence of solvents and reagents whose toxicological profile allows their use on living beings and on human beings in particular, without previously modifying the structural bonds of keratin and in particular in absence of modifications to the peptide bonds and disulfide bridges, which damage hair.

The (co)polymers according to the invention can constitute in themselves and thus provide a structural, protective, aesthetic or reconstructive function or they can constitute the substrate to fix to the keratin surface other molecules with function of functional agents such as: pigments and dyes, protective agents such as antioxidants, UV absorbers and antisolars or antibacterials, or chemical structures with volumizing, lubricating, detangling, glossing, coloring and similar action. The (co)polymers according to formula (I), further provided of functional agents, are therefore object of the present invention, as well as object of the invention are the polymer structures of formula (I) further provided with molecules with medicinal activity, by itself and for medical use.

A further object of the invention is therefore the presence of further functional groups on the (co)polymer structures of the invention that are useful in the anchorage of molecules or functional agents. In this embodiment the anchorage happens after the polymer synthesis.

A further object of the invention is a new methodology to anchor said functional agents, such as dyes, antioxidants, glossing agents, bulking agents, suntan filters, pigments, etc onto the polymer matrix.

A further object of the invention is the cosmetic use of polymeric structures of formula (I).

Another object of the invention are the cosmetic formulations containing such polymer matrix and the conditions and mode of use as innovative cosmetic product.

Another object of the invention is the synthesis of (co)polymers suitable for the above mentioned use.

### DESCRIPTION OF PREFERRED EMBODIMENTS

The (co)polymer structures of the invention have general formula:

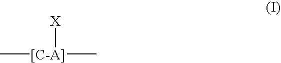

wherein [C-A] is a portion of a polymeric or copolymeric chain selected from an acrylic silicone, polyglycolic, polysaccharide polymeric structure or from a natural polymer,
and X is a functional group selected from one or more solfonic, carbossilic, amino acidic or epoxide groups to react at room temperature and in reaction conditions suitable for use on living beings and to form ionic or covalent bonds with keratin in absence of preemptive modifications of the polymer chain itself. According to the present invention, molecular weight of the polymers suitable for reacting with keratin structure is less in the range from 5.000 to 10.000, preferably from 10.000 to 100.000 Daltons.

According to another aspect of the invention, in the polymer chain the monomers provided with the functional group selected from sulphonic, carboxylic, amino acidic or epoxide groups represent at least 30, preferably at least 40 mole % and more preferably in the range from 50 and 90 mole % out of the total moles of monomers used for the polymer synthesis according to the invention.

In a preferred embodiment, molecular weight is in the range from 10.000 to 100.000 Daltons and the quantity in moles of the functionalized is in the range from 50% to 90% of the total moles of monomers used for the chain synthesis.

It was surprisingly discovered that the polymers having above mentioned features can react with keratin functional groups, such as polypeptidic groups and —NH₂, in water and at room temperature or anyway not higher than 45-50° C., which can be therefore obtained by a mild heating. Moreover, as already mentioned, the reaction between the polymers of present invention and keratin structure occurs in absence of activators and modificators of keratin structure.

Preferably, when X is —COOH or an epoxide group, the (co)polymer chain is selected from an acrylic and polyglycol structure, but such disclaimer, i.e. limitation, is not applied when the (co)polymer structure is also provided with molecules i.e. functional agents linked to the same, as hereafter better explained.

In one embodiment of the invention, the polymer structure presents a plurality of functionalized units of general formula

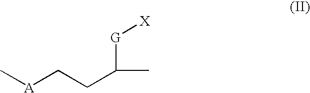

where X is selected, as mentioned above, from —SO₃H; —COOH; amino acidic groups and one epoxide group; G is a bond or is selected alternatively or together with A from acrylic and methacrylic, vinyl, styrene, butadiene, terpene residues and maleic anhydride and its derivatives.

The definition of G and A indicates and comprises in particular the following compounds:

G=Derivatives of acrylic, methacrylic, and itaconic acid and their methyl, ethyl esters and with C₃ to C₃₀ alkyl group, acryloyl morpholine and its derivatives, styrene, styrene with halogen groups in o-, m-, p-positions and their derivatives, vinyl chloride and its derivatives, vinyl acetate and its derivatives, acrylamide and its derivatives, N,N-dimethylacrylamide and its derivatives, N,N-diethylacrylamide and its derivatives, acrylamide substituted with C₃ to C₃₀ alkyl groups vinylpyrrolidones and its derivatives, butadiene and its derivatives, cyanoacrylates and cyanomethacrylates substituted with C₃ to C₃₀ alkyl groups, 2-acrylamide-2-methyl-1-propanesulfonic, furoic acid and its derivatives, vinylcarbazole and its derivatives, vilidene chloride and its derivatives, vinyl alcohol and its derivatives, terpenes and modified terpenes and maleic anhydride and its derivatives.
A=acrylic and methacrylic, itaconic acid and their methyl, ethyl esters and with alkyl group from C₃ to C₃₀, acryloyl morpholine and its derivatives, styrene, styrene with halogen groups in positions o-, m-, p- and its derivatives, vinyl chloride and its derivatives, vinyl acetate and its derivatives, acrylamide and its derivatives, N,N-dimethylacrylamide and its derivatives, N,N,diethylacrylamide and its derivatives, acrylamide with substituted C₃ to C₃₀ alkyl groups, vinylpyrrolidones and its derivatives, butadiene and its derivatives, cyanoacrylates and cyanomethacrylates, furoic acid and its derivatives, vinylcarbazole and its derivatives, vilidene chloride and its derivatives, vinyl alcohol, terpenes and modified terpenes and maleic anhydride and its derivatives.

Specifically, G, when different from a simple bond and from —CH₂— (alkenyl), and conforming with the above list, will have a double bond available to participate in the polymer synthesis reaction, as shown below:

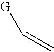

The polymer structure according to the invention is able to form ionic or covalent bonds with keratin at room temperature and under reaction conditions compatible with use on humans.

With the purpose of obtaining polymers which react with keratin under mild conditions, four types of functional groups have been identified, indicated by X in the general formula, that can form a stable chemical bond with hair:
1. sulfonic groups (derived from monomers carrying the sulfonic groups)
2. carboxyl groups (derived from the polymerization of functional monomers carrying one or more carboxyl groups
3. derived polymerizable amino acids (derived from amino acids that suitably functionalized carry a polymerizable group) and
4. epoxide groups (derived from monomers carrying an epoxide group).

The four functional groups can form strong bonds (ionic or covalent), though according to different mechanisms.

The polymers with sulfonic or carboxylic groups allow the polymer to form a bond with the keratin of the hair by ionic-covalent interactions.

The invention differs from all above mentioned documents of prior art. The document which is the closest to present invention is Application No. WO2007/072521 which, as already reported, is based on the formation of bonds between polymer and keratin by means of nucleophilic substitutions or of more traditional —SH bonds with preemptively treated keratin structure. The polymers of present invention, on the contrary, have functional groups which do not require to treat and therefore damage the keratin before the treatment, because they react by means of a condensation reaction forming an amidic or sulfonamidic bond and/or a ionic bond.

As hereafter specified, reactions occur in water or, in substantially aqueous solvents and in extremely mild conditions.

The principal advantages derived from the use of sulfonic and carboxylic groups is the facility with which interactions of ionic-covalent character can be created by mere contact of the polymer with the hair, because of the generous quantity of keratin functional groups that present partial or net charges. In this way a polymer is achieved that can bind to the keratin structure under mild conditions and that cannot be removed from the hair under normal conditions of washing.

In a first embodiment the polymers useful for the purpose of binding to keratin through predominantly ionic character, object of the present invention, are characterized by the presence of the —SO₃H group or —COON; their synthesis can be schematically shown as follows:

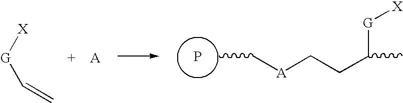

where X═—SO₃H; —COOH
and G and A have the above reported meaning, i.e. the polymer has a polyacrilic structure.

The same functionalization and the same purpose can be obtained with suitably activated linear or cyclical silicone polymers, polysaccharides and polymers of natural origin that can easily be functionalized with the active groups for the purpose described below, object of the present invention, to chemically bind the polymer structure to the hair.

When X is —COOH, monomers constituted by derived polymerizable amino acids, i.e. monomers derived from amino acid that when suitably functionalized carry a polymerizable group can be used advantageously for the synthesis of the (co)polymer structure of the invention.

The principal advantages derived from the use of derived amino acids can be attributed to their affinity for the (peptide) keratin structure of the hair, facility with which interactions of ionic-covalent character can be created by mere contact of the polymer with the hair, thus producing a polymer that can bind to the keratin structure and that cannot be removed simply by washing.

Their synthesis can be shown schematically as follows.

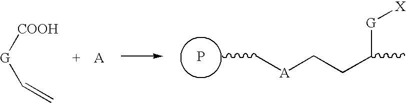

where G-COOH is a derivative of aspartic acid, glutamic acid, phenylalanine, glycine, histadine, isoleucine, lysine, leucine, methionine, asparagine, proline, glutamine, arginine, serine, threonine, valine, tryptophan, thyroxine. Such monomers have primarily been achieved by reaction of the amino acids with a suitable acrylic reagent (acrylic acid, acryloyl chloride, etc.) and where A has the above reported meaning.

According to another embodiment of the invention the binding functional groups of the copolymer structure to the keratin are epoxide groups and are inserted into the copolymer chain through monomers carrying the epoxide group.

The principal advantages derived from the use of monomers carrying epoxide function are attributed to their reactivity with the keratin (peptide) structure of the hair and specifically to the nucleophile function present on the keratin able to react with the oxyranic group forming covalent bonds under extremely mild conditions.

The synthesis of these copolymer structures is shown schematically as follows.

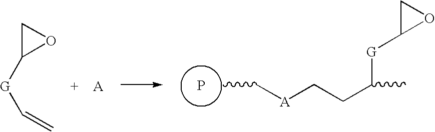

where G and A have the above reported meaning, i.e. that

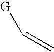

is selected from derivatives of acrylic and methacrylic, itaconic acid and their methyl, ethyl esters and with alkyl group from C₃ to C₃₀, acryloyl morpholine and its derivatives, styrene, styrene with halogen groups in positions o-, m-, p- and its derivatives, vinyl chloride and its derivatives, vinyl acetate and its derivatives, acrylamide and its derivatives, N,N-dimethylacrylamide and its derivatives, N,N-diethylacrylamide and its derivatives, acrylamide substituted with C₃ to C₃₀ alkyl groups, vinylpyrrolidones and its derivatives, butadiene and its derivatives, cyanoacrylates and cyanomethacrylates substituted with C₃ to C₃₀ alkyl groups, furoic acid and its derivatives, vinylcarbazole and its derivatives, vilidene chloride and its derivatives, vinyl alcohol and its derivatives, terpene and modified terpenes, and maleic anhydride and its derivatives.

A is selected from one or more acrylic and methacrylic, itaconic acid and their methyl, ethyl esters and with C₃ to C₃₀ alkyl group, acryloyl morpholine and its derivatives, styrene, styrene with halogen groups in positions o-, m-, p- and its derivatives, vinyl chloride and its derivatives, vinyl acetate and its derivatives, acrylamide and its derivatives, N,N-dimethylacrylamide and its derivatives, N,N,diethylacrylamide and its derivatives, acrylamide substituted with C₃ to C₃₀ alkyl groups, vinylpyrrolidones and its derivatives, butadiene and its derivatives, cyanoacrylates and cyanomethacrylates substituted with C₃ to C₃₀ alkyl groups, furoic acid and its derivatives, vinylcarbazole and its derivatives, vilidene chloride and its derivatives, vinyl alcohol and its derivatives, terpenes and modified terpenes and maleic anhydride and its derivatives.

The following Table lists some compounds useful in the synthesis of the polymers according to the invention.

TABLE 1

| | | |
|---|---|---|
| 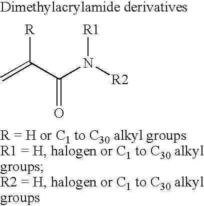 | 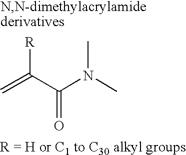 | 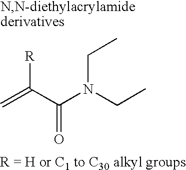 |
| 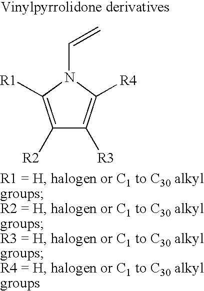 | 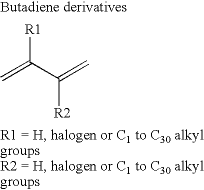 | 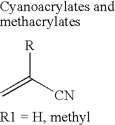 |
| 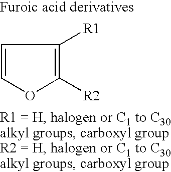 | 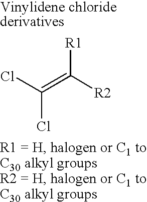 | 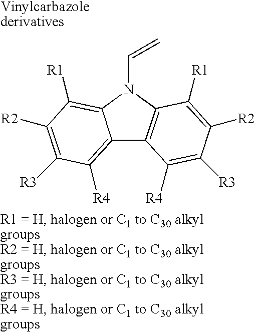 |
| 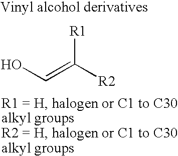 | 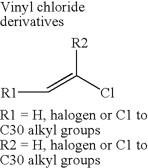 | 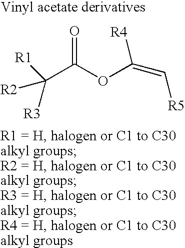 |
| 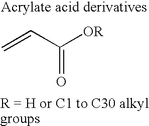 | 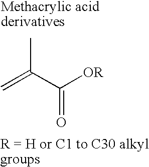 | 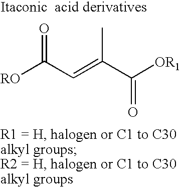 |
| 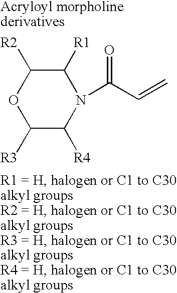 | 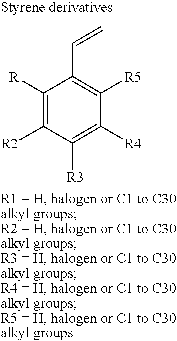 | |

A further advantage of the present invention is the possibility of covalent anchorage of molecules or functional agents on the (co)polymer achieved. In other words, through the (co)polymers of the invention it is possible to chemically bind functional agents or molecules (PF) to keratin, which meet the needs to protect or to modify the aspect of the keratin itself. The binding of such functional agents to the polymer chain itself can be achieved in different ways.

A classical way consists in reacting according to the canons of classical synthesis suitable reactive groups present on the polymer with some corresponding reagent on the PF that doesn't jeopardize the structure and functionality of the functional principle.

With the present invention a further way has been identified and experimented with success to functionalize the (co)polymer structure through the addition of at least one PF in suitable condition into the monomer mixture during radical polymerization.

Since the propagation of the polymer chain through free radical initiator doesn't happen in a linear way, but there are numerous kinds radicals present in the reaction environment reactive at the same time, it is possible through radical transposition that situations can occur at the same time in which a radical is produced on the propagation chain in and at the same time situated on the structure of the PF. In such a way the extinction of the two kinds of it radical through the formation of a covalent bond is an easy reaction by which to achieve an effective and practical method for the insertion of such PFs into the polymer chain.

The principal advantages derived from this invention are the covalent anchorage of the functional principle and the process of formation of the polymer in a single synthesis step, as in the scheme below.

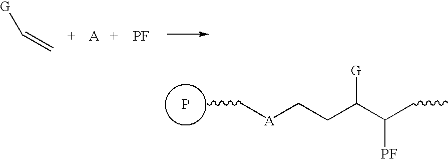

where G and A have the meaning reported above and where
PF indicates a molecule or functional agent, i.e. a compound having a practical function able to modify the characteristics of the polymer and the keratin to which the polymer must be fixed. Examples of functional agents of this type are: dyes, antioxidants, glossing agents, bulking agents, suntan filters, pigments.

The X group can be inserted subsequently, but it is preferably already present on the monomer, i.e. that the compound

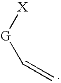

will be directly used in the polymer synthesis mixture, where X is selected from —SO₃H; —COOH; —H; and an epoxide group.

The invention will be now described more in detail with reference to the following examples, enclosed by way of illustration and not of limitation.

### Example 1

Formation of the copolymer carrying a group strongly interacting with the keratin of the hair and a functional agent (molecule).

Reaction (a)—Synthesis of Polymer A.

18 g of functional monomer (2-acrylamide-2-methyl-1-propanesulfonic) and 4 g of N,N-dimethylacrylamide and 300 mg of dye (Sudan III) in a suitable quantity of solvent (100 ml of THF) are added to a reaction vessel. After the addition of the initiator (40 mg of AIBN, i.e. a radical initiator: 2,2′-azo-bis-isobutyronitrile) is left under agitation for 24 h at 60° C.

On completion of the reaction, the polymer based on acrylic acid is precipitated therefore into ethyl ether and dried. A polymer is achieved provided by a G substituent constituted by —SO₃H, by the required chemical bond with the keratin, the polymer furthermore carrying directly in a side-chain a functional agent constituted by a dye (Sudan III).

Reaction (b)—Synthesis of Polymer B.

18 g of functional monomer (acrylic acid) and 4 g of acrylamide and 300 mg of dye (Sudan III) in a suitable quantity of solvent (100 g of THF) are added to a reaction vessel. After the addition of the initiator (400 mg of AIBN, i.e. a radical initiator 2,2′-azo-bis-isobutyronitrile) is left under agitation for 24 h at 60° C.

On completion of the reaction, the polymer based on acrylic acid is therefore precipitated into ethyl ether and dried.

The polymer formation reaction can in general involve various monomers, specifically those in the above-quoted lists, and to take place in different solvents and temperature conditions. The general scheme of the reaction is the following:

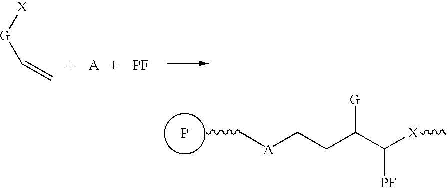

where G-X, A and PF have the meaning reported above.
Reaction (c)—Synthesis of the Copolymer C.

18 g of functional monomer (G-X=Glycylmethacrylate) and 4 g of acrylamide and 300 mg of dye (Sudan III) in a suitable quantity of solvent (100 g of THF) are added to a reaction vessel. After the addition of the initiator (400 mg of AIBN, i.e. a radical initiator, Azo-isobutyrronitrile) is left under agitation for 24 h at 60° C.

On completion of the reaction, the polymer based on acrylic acid is therefore precipitated into ethyl ether and dried.

The polymer formation reaction can in general involve various monomers, specifically those in the above-quoted lists, and to take place in different solvents and temperature conditions. The general scheme of the reaction is the following:

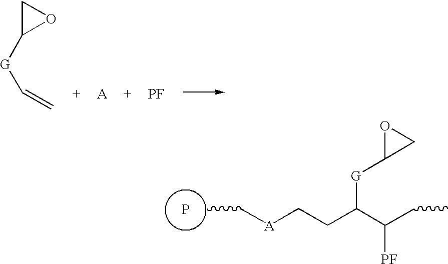

### Example 2

### Reaction of Anchorage of the Functionalized Polymer to the Hair

A solution of polymer (B) in which G-X is a —COOH group, carrying a functional principle (Sudan III) on a side-chain, is prepared by dissolving 100 mg of the polymer in 4 ml of H₂O.

Subsequently, 50 mg of a sample of blond human hair are immersed in the solution and allowed to react for 45 minutes at 55-60° C.

The desired reaction is that between the peptide bond and the amine group and the carboxylic groups present in the polymer. From such reaction the formation of new ionic-covalent bonds is achieved.

The hair, after the reaction (that simulates conditions analogous to those that are found at the hairdressers under the hair-drying helmet), was repeatedly washed with aggressive detergents (soap at pH 10) and with neutral detergents, simulating the repeated action of extended washings. It was then treated with solvents of various polarity and subjected, during such treatments, to ultrasounds. Despite such tests the sample remained clearly and firmly colored red, demonstrating the formation of the chemical bond as in the scheme below.

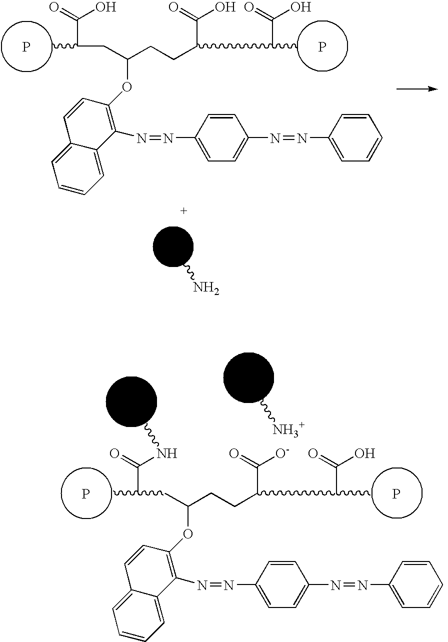

### Example 3

### Comparison

A sample of the same hair was treated in parallel under the same conditions of time and temperature, with the dye alone, giving a lock of red-colored hair. But in this case, the color was gradually but quickly lost during the washings, producing a lock of hair whose final appearance was the same as the initial.

### Example 4

### Reaction of Anchorage of the Functionalized Polymer to the Hair

A solution of polymer (A) in which G-X is a SO₃H group carrying a functional principle (Sudan III) on a side-chain, was prepared by dissolving 100 mg of polymer in 4 ml of H₂O.

Subsequently, 50 mg of hair was immersed in the solution and allowed to react for 45 min at 55-60° C. The desired reaction is that between the peptide bond and the amine group and the sulfonic groups present in the polymer. From such reaction the formation of new covalent bonds is achieved as in the scheme below:

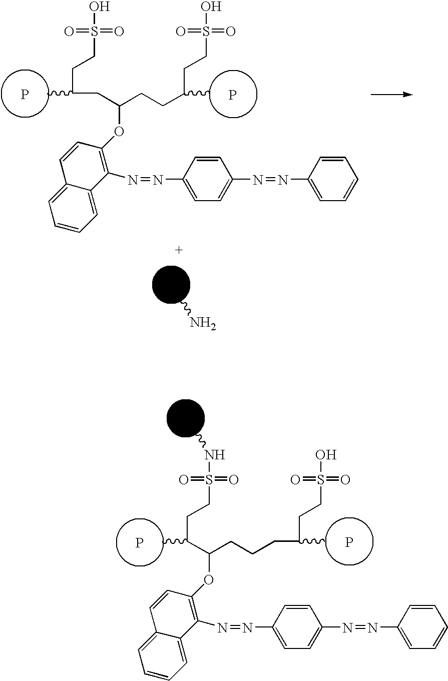

### Example 5

### Reaction of Anchorage of the Polymer Functionalized to the Hair

A solution of polymer (D) carrying a functional principle (Sudan III) on a side-chain, was prepared by dissolving 100 mg of the polymer in 4 ml of H₂O. Subsequently, 50 mg of hair was immersed in the solution and allowed to react for 45 min at 55-60° C. The desired reaction is that between the peptide bond and the amine group and the sulfonic groups present on the polymer. From such reaction the formation of new covalent bonds is achieved as shown here:

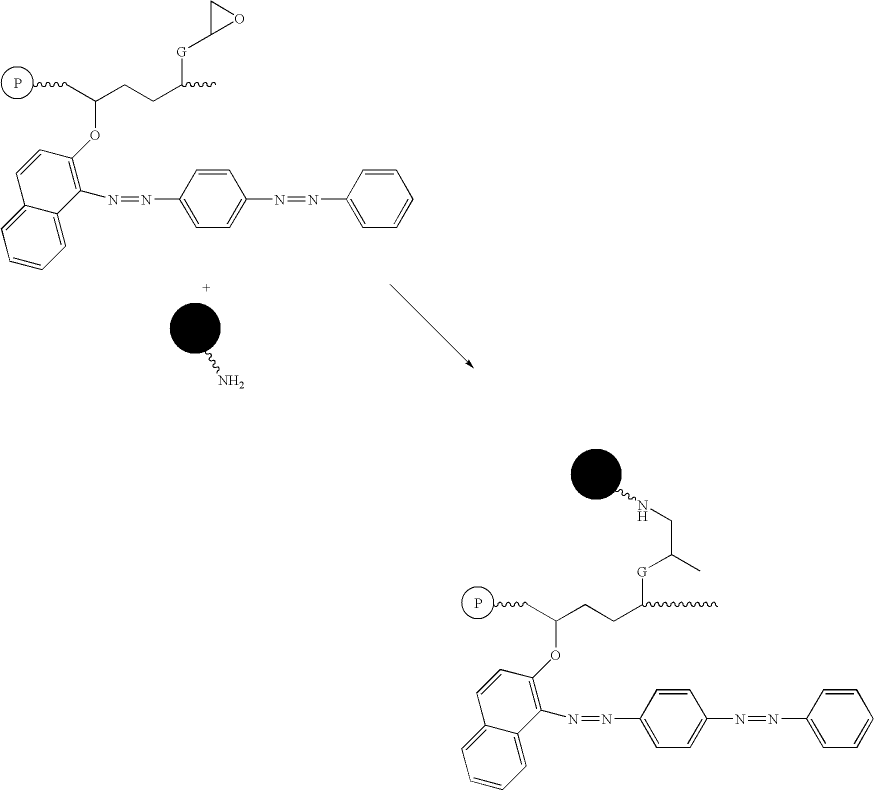

1. A polymer structure of general formula 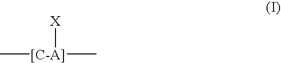
wherein [C-A] is a portion of a polymeric or co-polymeric chain selected from an acrylic silicone, polyglycolic, polysaccharide polymeric structure or from a natural polymer,
and X is a functional group selected from one or more sulphonic, carboxylic, amino acidic or epoxide groups to react at room temperature and in reaction conditions suitable for use on living beings and to form ionic or covalent bonds with keratin in absence of preemptive modifications of the polymer chain itself.

2. (Co)polymer structure according to claim 1, wherein the amount of monomers carrying a functional group X is at least 30 mole % out of the total moles of monomers used for the synthesis of said chain.

3. (Co)polymer structure according to claim 2, wherein the quantity of monomers carrying a functional group X is within the range from 50 mole % to 90 mole % out of the total moles of monomers used for the synthesis of said chain.

4. (Co)polymer structure according to claim 1, 2 or 3, comprising a plurality of functionalized units of general formula: 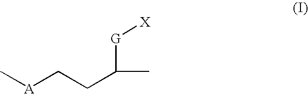
where X is selected from —SO₃H; —COOH and an epoxide group;
G and A are alternatively or together a bond, —CH₂, acrylic and methacrylic, vinyl, styrene, butadiene, terpene and maleic anhydride and its derivatives groups or residues,
and wherein
when X is —COOH, G-X is selected from —COOH and an amino acid.

5. Polymer structure according to claim 4, wherein said amino acid is selected from aspartic acid, glutamic acid, phenylalanine, glycine, histadine, isoleucine, lysine, leucine, methionine, asparagine, proline, glutamine, arginine, serine, threonine, valine, tryptophan, thyroxine.

6. (Co)polymer structure according to claim 4, wherein said acrylic and methacrylic, vinyl, styrene, terpene residues are selected from acrylic and methacrylic acid and their derivatives, itaconic acid and their methyl, ethyl esters and esters with a C₃ to C₃₀ alkyl group, acryloyl morpholine and its derivatives, styrene, styrene with halogen groups in o-, m-, p-positions and its derivatives, vinyl chloride and its derivatives, vinyl acetate and its derivatives, acrylamide and its derivatives, N,N-dimethylacrylamide and its derivatives, N,N,diethylacrylamide and its derivatives, acrylamide substituted with C₃-C₃₀ alkyl groups, vinylpyrrolidones and its derivatives, butadiene and its derivatives, cyanoacrylates and cyanomethacrylates substituted with C₃-C₃₀ alkyl groups, furoic acid and its derivatives, vinylcarbazole and its derivatives, vinylidene chloride and its derivatives, vinyl alcohol and its derivatives, terpenes and modified terpenes.

7. (Co)polymer structure according to any of claims from 1 to 6, further comprising one or more functional agents (PF) bound in a covalent way to said (co)polymer structure to modify the features of the same, said functional agents (PF) not being able to react with said functional groups (X) of said (co)polymer structure.

8. (Co)polymer structure according to claim 7, wherein said functional groups are selected from one or more coloring, antioxidant, glossing agents, bulking agents, suntan filters, pigments, antibacterial.

9. Use of the (co)polymer structures of general formula 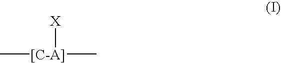
wherein [C-A] is a portion of a polymeric or co-polymeric chain selected from an acrylic silicone, polyglycolic, polysaccharide polymeric structure or from a natural polymer,
and X is a functional group selected from one or more sulfonic, carboxylic, amino acidic or epoxy groups to react at room temperature and in reaction conditions suitable for use on living beings and to form ionic or covalent bonds with keratin in absence of preemptive modifications of the polymer chain itself, or according to any of claims 2 to 8 for the treatment of hair, body hair, skin and nails.

10. A cosmetic formulation comprising a (co)polymer structure according to any claims 1 to 8 in combination with excipients, solvents and cosmetically acceptable carriers.

11. Cosmetic formulation according to claim 10, comprising two or more (co)polymer structures, wherein functional groups (X) present in said structures are selected so that they cannot react together under use conditions of the formulation.

12. A method for the treatment of keratin structures selected from hair, body hair, skin, mucosa and nails, characterized in applying to said structures a cosmetic formulation according to any of claims 1 to 8, or a cosmetic formulation according to any of claim 10 or 11, to bind in a ionic or covalent way the (co)polymer structure or structures contained in said formulation to said keratin structure in absence of preemptive modifications of the structural keratin bonds.

13. Method according to claim 12, wherein said covalent bond is formed through a condensation reaction among the functional groups on polymeric structure and free —NH₂ groups on keratin structure.

14. (Co)polymeric structure of formula (I), according to any claims 1 to 8, wherein said functional agents are formed by molecules with medical activity, for use in medical field.

15. A process for the synthesis of a (co)polymeric structure according to any of claims 1 to 8, characterized in that a compound of general formula 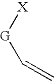
is reacted with a compound A
where X is selected from —SO₃H; —COOH; epoxide group,
and where G is a bond, i.e. together or alternatively to A is selected from —CH₂, acrylic and methacrylic, vinyl, styrene, butadiene, terpene groups and maleic anhydride and its derivatives.

16. A process according to claim 14, wherein said polymerization reaction is carried out in the presence of one or more molecules with functional features to insert in said polymeric chain said molecules as functional agents.
